(19) United States
(12) Patent Application Publication
(10) Pub. No.: US 2012/0027657 A1
(43) Pub. Date: Feb. 2, 2012

(54) CONTROL SYSTEM FOR UV-PCO AIR PURIFIER

(75) Inventors: Norberto O. Lemcoff, Simsbury, CT (US); Susan D. Brandes, South Windsor, CT (US); Leland G. Brandes, South Windsor, CT (US); Stephen O. Hay, Tolland, CT (US); Timothy N. Obee, South Windsor, CT (US); Catherine Thibaud-Erkey, South Windsor, CT (US)

(73) Assignee: CARRIER CORPORATION, Farmington, CT (US)

(21) Appl. No.: 12/864,638

(22) PCT Filed: Feb. 1, 2008
(86) PCT No.: PCT/US08/01420

§ 371 Date: Apr. 26, 2011

(51) Int. Cl. B01D 53/44; A61L 9/00; B01D 53/72

(52) U.S. Cl. 423/245.1; 422/120

(57) ABSTRACT

Ultraviolet photocatalytic oxidation (UV-PCO) air purification system includes controller that coordinates operation of photocatalytic reactor that removes volatile organic compounds from air and a regeneration mode that removes contaminants adsorbed in UV-PCO system. Controller coordinates operation of the regeneration mode and photocatalytic reactor so that when air purification system is turned on, the regeneration mode begins to operate before photocatalytic reactor is activated. The initial operation of the regeneration mode allows contaminants that have adsorbed in UV-PCO system to be removed before controller initiates a normal operation mode by activating photocatalytic reactor to cleanse the air.

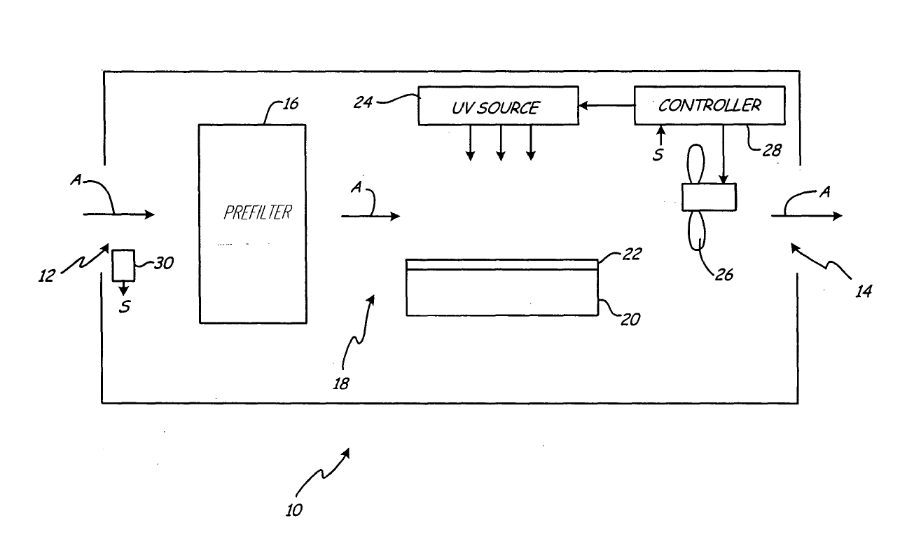

### BACKGROUND

This invention relates generally to the use of ultraviolet photocatalytic oxidation (UV-PCO) technology for decontamination of air in air purification systems. More specifically, the present invention relates to a control system and method for coordinating operation of components of the air purifier system when the system is first turned on after a period of inactivity.

Some buildings utilize air purification systems to remove airborne substances such as benzene, formaldehyde, and other contaminants from the air supply. Some of these purification systems include photocatalytic reactors that utilize a substrate or cartridge containing a photocatalyst. When placed under an appropriate light source, typically a UV light source, the photocatalyst interacts with oxygen and airborne water molecules to form active oxidation species such as hydroxyl radicals. The hydroxyl radicals then attack the contaminants and initiate an oxidation reaction that converts the contaminants into less harmful compounds, such as water and carbon dioxide. It is further believed that the combination of oxygen, water vapor, suitably energetic photons and a photocatalyst also generates an active oxygen agent like hydrogen peroxide. [W. Kubo and T. Tatsuma, Analytical Sciences, Vol. 20, 591-93 (2004)].

UV-PCO air purification systems are attractive because they convert volatile organic compounds (VOCs) to harmless compounds. The most common types of VOCs, which are pure hydrocarbons, are converted to water and carbon dioxide by the UV-PCO process. The typical operation of a UV-PCO involves periodic rather than continuous operation of the air purifier. The air purifier may be turned on by a timer, or by a control signal from an HVAC system. Typically, the photocatalytic reactor begins cleansing a flow of contaminated air when the UV-PCO air purifier is turned on.

### SUMMARY

The present invention is based upon the recognition that a UV-PCO air purifier may have been turned off for a considerable amount of time before it is turned on. During this off time period, considerable adsorption of volatile organic compounds may occur either on the photocatalyst surface of the reactor, or on an upstream filter. The concentration of volatile organic compounds can be excessively high. If this concentration of volatile organic compounds is present when the UV source of the photocatalytic reactor is turned on, the reaction occurring on the catalyst surface can lead to either incomplete oxidation or to generation of high molecular weight compounds that strongly adsorb on the photocatalyst surface and prevent other species from reaching the photocatalyst. As a result, the photocatalyst can suffer loses in activity, and the air purifier will suffer from reduced effectiveness.

In one embodiment, operation of the photocatalytic reactor is coordinated with a fan that is used to move air through the air purification system and the UV lamps that irradiate the catalyst. When the air purification system receives a command to begin operation after a period of inactivity, a controller coordinates operation of the fan and the photocatalytic reactor so that the fan operates for a period of time before the photocatalytic reactor is activated.

In another embodiment, the UV source is turned on under a minimum flow of clean air to quickly remove hydrocarbons such as toluene, xylene and ethylbenzene. In both embodiments, contaminants that adsorbed during the period of inactivity are removed from the system before the photocatalyst begins to convert volatile organic compounds in an airstream to harmless products.

### BRIEF DESCRIPTION OF THE DRAWINGS

The sole FIGURE is a block diagram schematically illustrating a photocatalytic air purification device.

### DETAILED DESCRIPTION

The FIGURE is a schematic diagram of air purifier system 10, which uses ultraviolet photocatalytic oxidation (UV-PCO) to remove contaminants from air. Air purifier system includes inlet 12, outlet 14, prefilter 16, photocatalytic reactor 18 (which includes substrate 20, photocatalytic coating 22, and UV source 24), fan 26 and controller 28.

Airstream A passes through prefilter 16 and then through photocatalytic reactor 18 and fan 26 to outlet 14. Prefilter 16 removes dust and particles from airstream A before they reach photocatalytic reactor 18. Prefilter 16 may contain a carbon filter to remove VOCs such as volatile silicon-containing compounds (VSCCs) from airstream A.

As airstream A passes through photocatalytic reactor 18, it comes in contact with photocatalytic coating 22. In the FIGURE, substrate 20 is illustrated schematically as a flat plate. In practice, substrate 20 can take a number of different forms, which may be configured to maximize surface area on which photocatalytic coating 22 is located (and thus maximize the surface area in which contact between photocatalytic coating 22 and airstream A can take place). One example is a honeycomb structure on which photocatalytic coating 22 is deposited and through which airstream A passes.

Ultraviolet radiation from UV source 24 is directed for and is absorbed by photocatalyst coating 22. The UV radiation causes photocatalyst coating 22 to interact with airborne water molecules to produce reactive species such as hydroxyl radicals, hydrogen peroxide, hydrogen peroxide radicals, and super oxide ions. These reactive species interact with VOCs in airstream A to transform VOCs into harmless byproducts such as carbon dioxide and water. Therefore, airstream A contains less contaminants as it exits system 10 through outlet 14 than it contained when it entered system 10 through inlet 12.

Controller 28 coordinates the operation of a regeneration mode and photocatalytic reactor 18. System 10 typically operates intermittently or periodically, rather than on a continuous basis. Controller 28, which may be, for example, a microprocessor based controller may receive commands from an HVAC system to initiate operation of air purifier system 10. Alternatively, controller 28 may be programmed to initiate and terminate operation of system 10 based upon a stored operating schedule or upon sensed parameters.

When system 10 is turned on, either by an external command received by controller 28 or as a result of a determination made by controller 28, the regeneration mode is operated for a period of time before air is purified or cleaned. Different regeneration modes can be used according to the type of contaminant present. For example, compounds which contain only hydrogen, carbon, and oxygen atoms usually only cause reversible damage. Example compounds include hydrocarbons such as toluene, xylene and ethylbenzene. On the other hand, volatile or semi-volatile organic compounds containing heteroatoms such as silicon, nitrogen, phosphorus and/or sulfur can lead to irreversible deactivation.

In one embodiment, the regeneration mode uses fan 26 to regenerate a surface in system 10. Fan 26 causes airflow through system 10 from inlet 12 to outlet 14. This airflow allows the concentration of VOCs that may have been adsorbed during the off period of system 10 to be moved through system 10 before UV source 24 is turned on and before photocatalyst coating 22 begins conversion of VOCs into harmless products.

The concentration of VOCs that may have accumulated on pre-filter 16 or on photocatalyst coating 22 during a period of inactivity of system 10 could adversely affect the operation of system 10 if UV source 24 is turned on immediately when system 10 is turned on. A high concentration of VOCs could result in incomplete oxidation or generation of high molecular weight compound that cause photocatalyst 22 to have reduced photocatalytic activity.

The period of time that fan 26 operates before UV source 24 is turned on can be a programmed time period within controller 28. A typical amount of time may be, for example, between about 5 minutes and about 10 minutes.

Controller 28 may include a real-time clock or other timer circuitry to determine how long system 10 was inactive before receiving a command to turn on. If the period of inactivity is relatively short, the time delay between operation of fan 26 and UV source 24 may be reduced, or may be eliminated in some circumstances. The amount of time required for fan 26 to move air through pre-filter 16 and photocatalytic reactor 18 may be controlled, therefore, as a function of the inactive period during which VOCs have been allowed to accumulate within system 10.

By not turning on UV source 24 when a high concentration of VOCs is adsorbed on photocatalyst coating 22, the deactivation of photocatalyst coating 22 is significantly reduced. The time delay between operation of fan 26 and UV source 24 does not significantly affect the overall operation of system 10 in its ability to remove contaminants from ambient air.

In another embodiment, the regeneration mode uses UV source 24 to regenerate photocatalyst coating 22 when compounds that cause reversible damage to photocatalyst coating 22 are present. For example, when photocatalyst coating 22 has been exposed to high levels of hydrocarbons (HCs), the hydrocarbons occupy some or all of the catalyst's active sites and are not efficiently and quickly removed from the surface just by purging it with clean air. UV light and a minimum flow of clean air removes these contaminates from photocatalyst coating 22.

Examples of high levels of hydrocarbons include when the total hydrocarbon concentration is 100 parts per billion or higher or when a specific hydrocarbon concentration (such as the toluene concentration) is 50 parts per billion or higher. Example hydrocarbons include hydrocarbons that are capable of chemisorbing onto a surface of system 10, such as toluene, xylene and ethylbenzene.

The minimum flow of clean air is the incidental or natural air flow through system 10 that is caused by the local heating of the air by UV source 24, and the clean air may be from the same source as the air which flows through system 10 during the operation of system 10. After flowing through system 10, the clean air may be directed to the building air supply or alternatively may be exhausted outside through vents.

The period of time that UV source 24 operates with a minimum flow of clean air before contaminated air is admitted into reactor 18 can be a programmed time period within controller 28. A typical amount of time may be, for example, between about 2 to about 8 hours.

Air purification system 10 may also include hydrocarbon (HC) measurement device 30, which is located upstream of photocatalytic reactor 18, such as at inlet 12. HC measurement device 30 measures the hydrocarbon concentration of airstream A. HC measurement device 30 may measure the total hydrocarbon concentration of airstream A, a specific hydrocarbon concentration, such as the toluene concentration, of airstream A, or any combination thereof. HC measuring device 30 may use gravimetric, thermal, resistive, electronic, magnetic, photolytic, optical or related sensing strategies, or any combination thereof, as the means of measuring the hydrocarbon concentration. HC measurement device 30 may send signal S, which represents the measured hydrocarbon concentration, to controller 28 to determine the appropriate operation procedure for system 10 after a prolonged period of inactivity. For example, the controller may only initiate a regeneration mode when a total hydrocarbon concentration of 100 parts per billion or higher is measured by HC measurement device 30, or when a specific hydrocarbon concentration, such as a toluene concentration, of 50 parts per billion or higher is measured by HC measurement device 30.

The regeneration mode used is based upon the environment in which the system is installed. Some environments may require using a plurality of regeneration modes. For example, in one system a regeneration mode performed by running a fan to remove VOCs is performed everyday while a regeneration mode performed by using a UV light source to remove hydrocarbons is performed on weekends. In another example, two different regeneration modes are performed in series in a system, such as regenerating a surface by removing VOCs followed by regenerating the same or different surface in the system by removing hydrocarbons.

Although the present invention has been described with reference to preferred embodiments, workers skilled in the art will recognize that changes may be made in form and detail without departing from the spirit and scope of the invention.

1. An air purification system comprising:
an inlet;
an outlet;
a photocatalytic reactor for removing volatile organic compounds (VOCs) from air; and
a controller for coordinating operation of the system so that upon the system beginning operation, the controller causes the system to operate in a regeneration mode for a time period to remove contaminants adsorbed during non-operation of the system before the controller initiates a normal operation mode by activating the photocatalytic reactor to cleanse the air.

2. The air purification system of claim 1, and further comprising a fan for causing air to enter the system through the inlet, to move through the photocatalytic reactor, and to exit the system through the outlet during the regeneration mode.

3. The air purification system of claim 2, wherein the time period is about 5 minutes to about 10 minutes.

4. The air purification system of claim 2, wherein during the regeneration mode the controller causes the fan to operate for a time period that is a function of a time duration of inactivity of the photocatalytic reactor prior to beginning operation of the fan.

5. The air purification system of claim 1, wherein during the regeneration mode the controller causes a UV source to remove hydrocarbons from a surface of the air purification system.

6. The air purification system of claim 5, wherein the time period is about 2 hours to about 8 hours.

7. The purification system of claim 5, and further comprising a hydrocarbon measurement device for measuring hydrocarbon concentration and wherein the controller coordinates the operation of the regeneration mode and the normal operation mode as a function of the hydrocarbon concentration.

8. The air purification system of claim 7, wherein the hydrocarbon concentration is total hydrocarbon concentration, and wherein the controller causes the system to operate in a regeneration mode when the total hydrocarbon concentration is about 100 parts per billion or higher.

9. The air purification system of claim 7, wherein the hydrocarbon concentration is concentration of a specific hydrocarbon, and wherein the controller causes the system to operate in a regeneration mode when the specific hydrocarbon concentration is about 50 parts per billion or higher.

10. The air purification system of claim 9, wherein the specific hydrocarbon is capable of chemisorbing on a surface of the air purification system.

11. The air purification system of claim 9, wherein the specific hydrocarbon is selected from the group consisting of toluene, xylene and ethylbenzene.

12. A method of removing volatile organic compounds (VOCs) from air, the method comprising:
regenerating a surface of an air purification system for an initial time period to dissipate contaminants accumulated during non-operation of a photocatalytic reactor; and
activating the photocatalytic reactor following the initial time period.

13. The method of claim 12, wherein regenerating a surface comprises directing air through the photocatalytic reactor for an initial time period to dissipate VOCs.

14. The method of claim 13, wherein the initial time period is about 5 to about 10 minutes.

15. The method of claim 13, wherein the initial time period is a function of a time duration of inactivity of the photocatalytic reactor.

16. The method of claim 12, wherein regenerating the surface comprises exposing the surface to UV light, and directing clean air through the photocatalytic reactor for a period of time to dissipate hydrocarbons.

17. The method of claim 16, and further comprising:
measuring total hydrocarbon concentration; and
regenerating the surface when the measured total hydrocarbon concentration is about 100 parts per billion or higher.

18. The method of claim 16, and further comprising:
measuring a specific hydrocarbon concentration; and
regenerating the surface when the measured specific hydrocarbon concentration is about 50 parts per billion or higher.

19. The method of claim 18, wherein the specific hydrocarbon concentration is a concentration of a hydrocarbon that is capable of chemisorbing on a surface of the air purification system.

20. The method of claim 19, wherein the hydrocarbon is selected from the group consisting of: toluene, xylene, and ethylbenzene.

21. An air purification system comprising:
an inlet;
an outlet;
a photocatalytic reactor for removing volatile organic compounds (VOCs) from air;
a prefilter for removing particulates from the air, the prefilter at a location upstream of the photocatalytic reactor; and
a controller for coordinating operation of the system so that upon the system beginning operation, the controller causes the system to operate in a regeneration mode for a time period to remove contaminants adsorbed during non-operation of the system before the controller initiates a normal operation mode by activating the photocatalytic reactor to cleanse the air.

22. The air purification system of claim 21, and further comprising a fan for causing air to enter the system through the inlet, to move through the photocatalytic reactor, and to exit the system through the outlet during the regeneration mode.

23. The air purification system of claim 22, wherein the time period is about 5 minutes to about 10 minutes.

24. The air purification system of claim 23, wherein during the regeneration mode the controller causes the fan to operate for a time period that is a function of a time duration of inactivity of the photocatalytic reactor prior to beginning operation of the fan.

25. The air purification system of claim 21, wherein during the regeneration mode the controller causes a UV source to operate to remove hydrocarbons (HCs) from a surface of the air purification system.

26. The air purification system of claim 25, wherein the time period is about 2 to about 8 hours.
